(19) United States
(12) Patent Application Publication
(10) Pub. No.: US 2011/0034551 A1
(43) Pub. Date: Feb. 10, 2011

(54) METHODS OF INCREASING SARCOSINE LEVELS FOR TREATING SCHIZOPHRENIA

(75) Inventor: Robert Nicholas McBurney, Newton, MA (US)

Correspondence Address: K&L Gates LLP, STATE STREET FINANCIAL CENTER, One Lincoln Street, BOSTON, MA, 02111-2950, US

(73) Assignee: BG MEDICINE, INC., Waltham, MA (US)

(21) Appl. No.: 12/678,614

(22) PCT Filed: Sep. 18, 2008
(86) PCT No.: PCT/US08/76842

§ 371 Date: Oct. 19, 2010

Related U.S. Application Data
(60) Provisional application No. 60/973,674, filed on Sep. 19, 2007.

(51) Int. Cl. A61K 31/195; A61K 31/216; A61K 31/192; A61P 25/18

(52) U.S. Cl. 514/533; 514/543; 514/571; 514/563

(57) ABSTRACT

Methods for increasing sarcosine levels in a patient are provided. The methods include activating the PPARα receptor. Increasing sarcosine levels can be used, for example, as part of a treatment for schizophrenia.

### FIELD OF THE INVENTION

The present invention relates to methods for increasing sarcosine levels, and their use in treating psychiatric and neurological diseases and disorders, such as schizophrenia.

### BACKGROUND OF THE INVENTION

Schizophrenia affects approximately 0.5% of the US population and a similar percentage of the world population. Schizophrenia is one of the most severe and debilitating of the major psychiatric diseases. It usually starts in late adolescence or early adult life and often becomes chronic and disabling. Men and women are at equal risk of developing this illness; however, most males become ill between 16 and 25 years old, while females develop symptoms between 25 and 30. People with schizophrenia often experience both “positive” symptoms (e.g., delusions, hallucinations, disorganized thinking, and agitation) and “negative” symptoms (e.g., lack of drive or initiative, social withdrawal, apathy, and emotional unresponsiveness).

Current medicines for treating schizophrenia neither cure the disease nor even successfully treat all symptoms in patients who respond to treatment. Currently-used antipsychotic medicines share, as a common mechanism, the ability to antagonize dopamine D2 receptors in the mammalian brain and it is widely assumed that this activity contributes to their efficacy against the positive symptoms of schizophrenia (Seeman & Lee, Science 188:1217-9 (1975); Creese, et al., Science 192:481-3 (1976)). However, the efficacy of these currently-used medicines on the negative and cognitive symptoms of this disease is not optimal (Cassens et al., Schizophr. Bull. 16:477-99 (1990); King, Acta Psychiatr. Scand. Suppl. 380:53-8 (1994)).

Another treatment for schizophrenia that has been investigated involves the administration of sarcosine (N-methylglycine). Intake of 2 g/day sarcosine as add-on therapy to certain antipsychotics (but not clozapine (Lane, et al., Biol. Psychiatry, 60:645-9 (2006)) in schizophrenia gives additional reductions in both positive and negative symptomatology as well as the neurocognitive, general psychiatric and depressive symptoms that are common to the illness (Lane, et al., Arch. Gen. Psychiatry 62:1196-204 (2005); Heresco-Levy, Evid. Based Ment. Health 9:48 (2006); Lane, et al., Biol. Psychiatry (2007)). Sarcosine is also under investigation for the possible prevention of schizophrenic illness during the prodromal stage of the disease.

### SUMMARY OF THE INVENTION

The present invention provides methods for increasing sarcosine levels in a patient who may benefit from an increase in sarcosine levels. The methods include increasing sarcosine levels by activating peroxisome proliferator activated receptor type α (PPARα).

An increase in sarcosine levels can be used to promote NMDA receptor activity in a patient who would benefit from increased NMDA receptor activity. Thus, the invention also provides methods for promoting NMDA receptor activity by activating PPARα and increasing sarcosine levels.

Deficiencies in NMDA receptor activity are associated with psychiatric and neurological diseases and disorders including schizophrenia. Accordingly, the invention also provides methods for treating schizophrenia by promoting NMDA receptor activity through activation of PPARα and elevation of sarcosine levels. Activation of PPARα can be combined with other treatments for schizophrenia. For example, the treatment method can include both activating PPARα and antagonizing dopamine D2 receptors.

PPARα activation can be effected by any medically suitable method, such as by administering one or more compounds selected from the group consisting of clofibrate, gemfibrozil, ciprofibrate, bezafibrate, fenofibrate, simfibrate, and clofibride, and pharmaceutically acceptable salts thereof. The method can include administering, for example, a compound that activates both PPARα and other receptors, such as PPARγ, or it may be a “pure” PPARα agonist that does not have a comparable effect on other human PPAR receptors.

### BRIEF DESCRIPTION OF THE DRAWINGS

FIG. 1 is a flowchart showing a general approach for identifying unknown target compounds using GC/MS analyses.

FIG. 2 is a flowchart showing a general approach for identifying unknown target compounds using Polar LC/MS analyses.

### DETAILED DESCRIPTION OF THE INVENTION

The present invention benefits from the discovery that PPARα agonists increase plasma sarcosine concentrations. Indeed, the present inventor has demonstrated that two different PPARα agonists with different chemical structures are each capable of inducing several fold increases in circulating sarcosine concentrations. Increasing sarcosine concentrations can be used, for example, as part of a treatment for schizophrenia and to promote NMDA receptor activity. Because different PPARα agonists are each capable of increasing sarcosine levels, it is anticipated that any PPARα agonist, such as clofibrate, gemfibrozil (Lopid®), ciprofibrate (Modalim®), bezafibrate (Bezalip®), fenofibrate (TriCor®), etofibrate, simfibrate, clofibride, or a pharmaceutically acceptable salt thereof, can be used in the present invention. The agonists can be administered alone or in combination with other PPARα agonists. In addition, PPARα agonists can be used in combination with other therapies for the treatment of psychiatric and neurological diseases. When multiple compounds are used, they can be administered simultaneously, or sequentially in any order.

As the methods of the invention permit an increase in sarcosine concentrations, the methods can be used to inhibit the type 1 glycine transporter, GLYT1. GLYT1 removes glycine from the extracellular space in the region of NMDA receptors (Betz, et al., Biochem. Soc. Trans. 34:55-8 (2006)). Sarcosine can inhibit glycine transport in rat brain aggregates with an IC50 of 13 micromolar (Atkinson, et al., Mol. Pharmacol. 60:1414-20 (2001)). In other studies, sarcosine was shown to have an IC50 of around 100 micromolar for inhibition of glycine transport.

By inhibiting GLYT1, the invention permits an increase in local glycine concentrations and an associated increase in NMDA receptor activity. Glycine is a co-agonist of glutamate at the NMDA receptor, increasing the affinity of the receptor for the endogenous agonist glutamate (Johnson & Ascher, Nature 325:529-31 (1987); Kleckner & Dingledine, Science 241:835-7 (1988); Forsythe et al., J. Neurosci. 8:3733-41 (1988); Foster & Kemp, Nature 338:377-8 (1989)). Hence, a strategy which increases the activation of the glycine co-agonist receptor site (glycine B site) on the NMDA receptor also increases the response of the NMDA receptor to stimulation at the glutamate receptor site on the NMDA receptor. GLYT1-specific inhibitors have been found to enlarge NMDA receptor mediated ionic currents in spinal cord (Lim, et al., J. Neurophysiol. 92:2530-7 (2004)). Similarly, in hippocampal slice preparations, partial inhibition of GLYT1 caused a facilitation of NMDA receptor-mediated responses, resulting in enhanced long-term potentiation (Martina, et al., J. Physiol. 557:489-500 (2004); Igartua, et al., Neuropharmacology 52:1586-95 (2007)). Indeed, the inhibition of glycine transport by sarcosine has an (indirect) potentiating effect on neuronal activity mediated by the NMDA receptor (Martina, et al., J. Physiol. 557:489-500 (2004)).

The present invention provides additional treatment options for psychiatric and neurologic disorders whose treatment can benefit from increased NMDA receptor activity. Schizophrenia, for example, involves hypofunction of a subpopulation of cortico-limbic NMDA receptors. Low doses of the drug ketamine, which inhibits the function of the NMDA receptor by blocking its ion channel, replicate in normal volunteers the positive, negative and cognitive symptoms of schizophrenia as well as associated physiologic abnormalities, such as eye tracking (Krystal et al., Arch. Gen. Psychiatry 51:199-214 (1994); Adler et al., Am. J. Psychiatry 156:1646-9 (1999); Avila et al., Am. J. Psychiatry 159:1490-6 (2002)). Furthermore, genetic studies have identified putative risk genes for schizophrenia. The products of these genes, including D-amino oxidase, proline oxidase and neuregulin, have been shown to influence NMDA receptors (Coyle, Neurotox. Res. 10:221-33 (2006)). A treatment that enhances NMDA receptor activity should prove useful for treatment of the complex symptoms that define schizophrenia. Indeed, intake of 2 g/day sarcosine as add-on therapy to certain antipsychotics (but not clozapine (Lane, et al., Biol. Psychiatry, 60:645-9 (2006)) in schizophrenia gives additional reductions in both positive and negative symptomatology as well as the neurocognitive, general psychiatric and depressive symptoms that are common to the illness (Lane, et al., Arch. Gen. Psychiatry 62:1196-204 (2005); Heresco-Levy, Evid. Based Ment. Health 9:48 (2006); Lane, et al., Biol. Psychiatry (2007)).

PPARα agonists can be used in combination with other therapies, such as dopamine D2 antagonists, for the treatment of schizophrenia or other psychiatric or neurological diseases. Suitable dopamine D2 receptor antagonists include, for example, amisulpride, benperidol, chlorpromazine, clozapine, flupentixol, fluphenazine, haloperidol, levopromazine, olanzapine, pericyazine, perphenazine, pimozide, prochlorperazine, promazine, quetiapine, remoxipride, risperidone, sertindole, sulpiride, trifluoroperazine, thioridazine, thiothixene, ziprasidone, and zotepine, and pharmaceutically acceptable salts thereof.

### Modes of Administration

Oral dosage forms are generally the most convenient for administration, and are readily available for PPARα agonists and for other compounds that can be administered in addition to a PPARα agonist, such as a dopamine D2 antagonist. Nevertheless, the invention is not so limited.

Accordingly, pharmaceutical compositions can be formulated for delivery by any available route including, but not limited to, parenteral (e.g., intravenous), intradermal, subcutaneous, oral (e.g., inhalation), transdermal (topical), transmucosal, rectal, and vaginal. Pharmaceutical compositions typically include an active compound or salt thereof, or a related compound or analog, in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. Formulations for oral delivery may advantageously incorporate agents to improve stability within the gastrointestinal tract and/or to enhance absorption.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

The amount of PPARα agonist used according to the present invention will depend on several factors such as the activity of the specific compound employed; the age, body weight, general health, gender, and diet of the subject; the seriousness of the psychiatric or neurological disorder; the individual response of the patient; the kind of formulation; and the route of administration. A therapeutically effective amount of a PPARα agonist can range from about 0.01 to about 500 mg/kg body weight. The PPARα-agonist can be provided in a single dose or can be divided into multiple daily doses, e.g., 2, 3, 4, or 5 times daily. A dose can contain from about 0.01 to about 500 mg/kg body weight, in particular about 0.01, 0.05, 0.1, 0.5, 1.0, 1.5, 2, 2.5, 5, 7.5, 10, 15, 20, 25, 50, 75, 100, 150, 200, 250, 300, 350, 400, 450, or 500 mg/kg body weight.

The pharmaceutical composition can be administered at various intervals and over different periods of time as required. For certain conditions it may be necessary to administer the therapeutic composition on an indefinite basis to keep the disease under control. The skilled artisan will appreciate that certain factors can influence the dosage and timing required to effectively treat a subject, including but not limited to the severity of the disease or disorder, previous treatments, the general health and/or age of the subject, and other diseases present. Generally, treatment of a subject in accordance with the present invention can include a single treatment or, in many cases, can include a series of treatments.

Pharmaceutical compositions can be included in a container, pack, or dispenser together with instructions for administration.

Compounds may be administered concurrently with an additional agent useful for treatment of the psychiatric or neurological disorder. Many such agents are known in the art and include a wide variety of typical and atypical anti-psychotic agents. In addition, the compounds may be administered concurrently with compounds useful for ameliorating the side effects of anti-psychotic agents. See, for example, Hardman, J. G., et al., (eds.) Goodman & Gilman's The Pharmacological Basis of Therapeutics, 10th edition, McGraw Hill, 2001, for discussion of numerous agents useful for the foregoing purposes. The concurrently administered compounds may be administered to the subject separately or may be formulated together.

### Example 1

### PPARα Agonists Increase Plasma Sarcosine Concentration

### Test Articles:

Fenofibrate (Sigma F-6020, Lot. 064K1584) and MPC-1 (Mitsubishi Pharma Corporation, Lot. F) were used in this study. These test substances were stored at room temperature.

### Animals:

Seventy-three male Crj:CD(SD)IGS rats (4.5 weeks old) were quarantined and acclimated for seven days after receipt. Two days before the initiation of dosing (Day −2), rats which had no abnormalities in clinical observation and body weight during the quarantine and acclimation period were selected and assigned to 5 groups using the stratified-by-weight randomization method to yield approximately the same mean body weights amongst the study groups.

### Housing and Maintenance Conditions:

Throughout the study period, including the quarantine and acclimation periods, the animal room was set to maintain temperature and relative humidity at 22±3 degrees Celsius and 55±20%, respectively, with air changes 10-20 times/hour and a 12 hour artificial light cycle.

Rats were housed in stainless steel cages (275 W×370 D×210 H mm in size). Three rats were housed per cage. The cages and feeders were autoclaved and were replaced once a week. A certified rodent diet irradiated with y rays was supplied ad libitum. Tap water filtered through a 5 μm filter and irradiated with UV was supplied ad libitum through a water-supply system.

### Dosing Method and Preparation of Dosing Suspension:

The rats were dosed by oral gavage using a disposable stomach tube connecting to a syringe, at the dose volume of 5 mL/kg. The dosing was conducted once daily for 28 days. A control group was dosed with the vehicle only. Individual doses were calculated based on the most recently recorded body weights.

Preparation of dosing suspensions was as follows. The specified amount of test substance for each dose level was weighed and crushed with an agate mortar and a pestle. A few drops of 0.5% hydroxypropylmethylcellulose (HPMC) solution (HPMC: Sigma, lot No. 093K0622) were added to the test substance in the mortar and mixed well with the pestle until a smooth suspension was formed. This suspension was transferred to a graduated cylinder which volume was more than the desired volume. The vehicle was added to achieve the desired volume. After ultrasonication (room temperature, 5 min), the required volume of each dose suspension was dispensed into sterilized polypropylene tubes for daily dosing. The dose suspensions were stored at 4° C. The preparations were performed once a week and the dosing suspensions were used within 8 days after the each preparation.

### Study Design:

Study design was as shown in Table 1. Animals were dosed daily for 28 days with Test Articles at the doses shown. The initiation of dosing was designated as Day 1.

TABLE 1
Study Design

| Group | | Dose | Number of |
|---|---|---|---|
| No. | Test Article | (mg/kg) | Animals |
| 1 | Vehicle (0.5% HPMC) | 0 | 15 |
| 2 | Fenofibrate | 100 | 12 |
| 3 | Fenofibrate | 300 | 15 |
| 4 | MPC-1 | 15 | 12 |

### Observations and Examinations:

Clinical signs of all rats were observed twice daily (before and after dosing) during the dosing period and once daily during the others periods. The body weights of all rats were measured on Day 1, Day 6, Day 13, Day 20, Day 27 and Day 29. Food consumption of each cage was measured as the gross weight on Day 1, Day 6, Day 9 Day 13, Day 16, Day 20, Day 24 and Day 29. Individual food consumption data of each day was calculated from the gross weight.

### Blood and Plasma Collection:

At sacrifice, Day 29, animals were anesthetized with sodium thiopental and blood samples were collected from the aorta abdominalis and transferred in blood collection tubes containing serum separator gels for serum separation or containing lithium heparin and protease inhibitor (100 μL of a stock solution created by adding 1 bottle of protease inhibitor (Sigma P-2714) to 2.2 mL of phosphate buffered saline) for plasma separation. Plasma was obtained by centrifugation (3000 rpm, 10 min). An aliquot of the plasma layer was transferred into a 1 mL tube, frozen immediately using liquid nitrogen and stored at −80° C.

### Bioanalytical Measurements:

### Targeted GC/MS:

The aliquoted plasma samples were thawed and extracted with methanol to create a plasma methanol extract. The methanol was evaporated from the plasma methanol extract under nitrogen. 10 μL of a 250 ng/μL standard of cholic acid D4 and alanine-D4 in pyridine was added. Before capping the autosampler vial, 30 μL ethoxyamine hydrochloride solution in pyridine was added and the sample was incubated at 40° C. for 90 minutes.

After the oximation, the remaining Internal Standards were added, 10 μL of a 250 ng/μL standard of difluorobiphenyl, dicyclohexylphtalate and trifluoroacetylanthracene in pyridine, and the sample was silylated by adding 100 μL MSTFA and heating for 90 minutes at 40° C. Before injection in the GC, the prepared samples were centrifuged for 20 minutes at 3500 rpm.

Based on the total number of samples, the batches of samples were set up according to a batching scheme. Each sample was analyzed in duplicate. The samples, sorted in batches, were stored in labeled boxes at −80° C. until analysis. Each batch is analyzed in one analytical run.

GC/MS analysis employed an Agilent 6890 N gas chromatograph equipped with a PTV (programmed temperature vaporizer) injector and a CTC Analytics Combi-Pal autosampler. For detection, an Agilent 5973 Mass Selective Detector is used. The system was controlled by Enhanced Chemstation G1701CA Version D.01.02 software.

All compounds eluting from the GC/MS column were detected in full scan mode. Each peak was characterized by its retention time and a number of fragments (m/z values). The amount of data (i.e. number of variables) was reduced by applying a target processing procedure. Quality Control of the analysis and the data was performed in several steps during the workflow. At the various steps in the sample preparation and analysis, one or more Internal Standards were added in order to monitor the quality of the data after analysis. After each batch, the response of the Internal Standards after initial correction for dicyclohexyl phthalate (DCHP) Internal Standard was evaluated in all samples. If the corrected peak area of each Internal Standard deviated less than 20% from the batch mean, the batch was approved. If for one or more samples the deviation was more than 20%, then these samples were reanalyzed in the next batch.

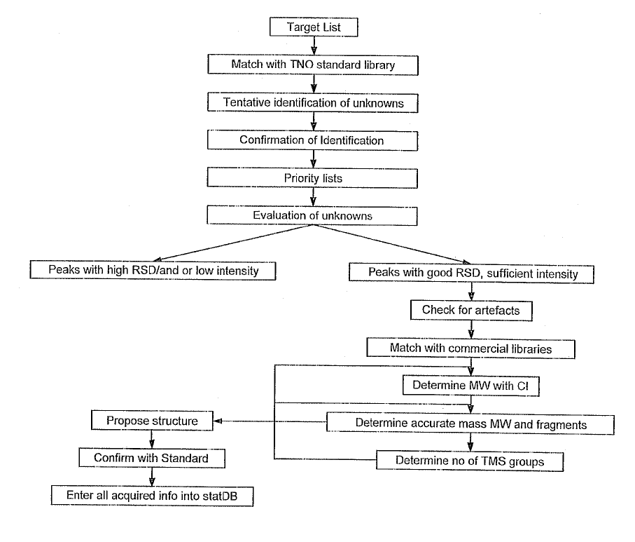

A general overview of all the steps that were taken to identify priority peaks is shown in FIG. 1.

The first identification step was matching of the target compound list with the reference standard database. A number of compounds from the target list were identified and confirmed by analyzing the standards in the same batch with a study sample. The unidentified spectral peaks prioritized from statistical analysis were first evaluated by inspecting the raw data. After this, additional identification methods were selected, depending on the individual (to be identified) compounds. For some compounds, hits were found in commercial spectral libraries and no additional experiments were required for identification. For other compounds chemical ionization, accurate mass determination or other derivatization experiments were performed.

### Derivatized Polar LC/MS:

For the Polar LC/MS platform, each study sample was divided into two analysis samples. These duplicate samples were derivatized separately and injected one after the other in the measurement phase of the workflow. To 85 μL of plasma methanol extract in a small Eppendorf® vial, 10 μL additional Internal Standard solution was added and the sample was vortexed briefly. The Internal Standard solution contained Cre-d3, Met-d4, Mhi-d3 (at 1 μg/mL) and Ala-d3 (at 2 μg/mL). After addition of dithiothreitol (DTT) solution and deproteinization of the sample, the supernatant was lyophilized. The samples were then derivatized with HCl-butanol at 65° C. The excess of the reagent was removed by lyophilization. The sample was reconstituted in an aqueous solution of DTT containing underivatized Tyrosine D7 as an Internal Standard.

The samples were derivatized in lots based on the total number of study samples. Each lot of samples was divided into a number of batches for analysis. In addition, each batch of samples contained a number of Quality Control samples which were prepared in the manner described above from a single pool of starting plasma. As outlined above each sample was analyzed in duplicate. The samples, sorted in batches, were stored at −80° C. until analysis. Each batch was analyzed in one analytical run.

A Varian/Chrompack Inertsil 5 μm ODS-3 100*3 mm column with a Varian/Chrompack R2 10×2 mm i.d. guard column was used in these analysis. A binary phase, 35 min. linear LC gradient was used. Mobile phase A contained 0.1% formic acid and Mobile phase B contained 80% Acetonitrile in 0.1% formic acid. The column temperature was adjusted just above room temperature to insure consistent chromatography. The injected volume was 10 μL.

A Thermo LTQ ion trap mass spectrometer was in this study for mass spectrometric profiling and determination of relative quantification.

All compounds eluting from the LC column were detected with a mass spectrometer in full scan mode. Each peak was characterized by its mass-to-charge ratio (m/z values) and its retention time. The number of peaks was reduced by applying a target processing procedure after which each compound in the chromatogram was, in most cases, represented by only one entry in the peak table.

After each batch, the raw peak area (response) of all the Internal Standards in all the samples was checked as well as the RSD (relative standard deviation) of the normalized peak area (relative response) of four amino acids present in the quality control samples. The raw data of the Internal Standards did not deviate by more than 25% from the batch average. The RSD of the normalized peak area of the four amino acids did not exceed 20%. This check was performed before starting the following batch.

After completion of the analysis of all batches, the peaks of all compounds present in the target table were first integrated using standard integration settings (expected retention time, baseline, peak width, etc.). For all targets, the deviation of the peak area from the mean (of all study and Quality Control samples) was calculated. For peaks which exhibited a large deviation in their peak area from the mean, peak integration was performed manually.

The quality control of the complete dataset was performed based on the Internal Standard response in all the samples except blanks and the check of relative standard deviations of all the target compounds in the Quality Control samples. These results were compared for consistency with those observed in previous studies.

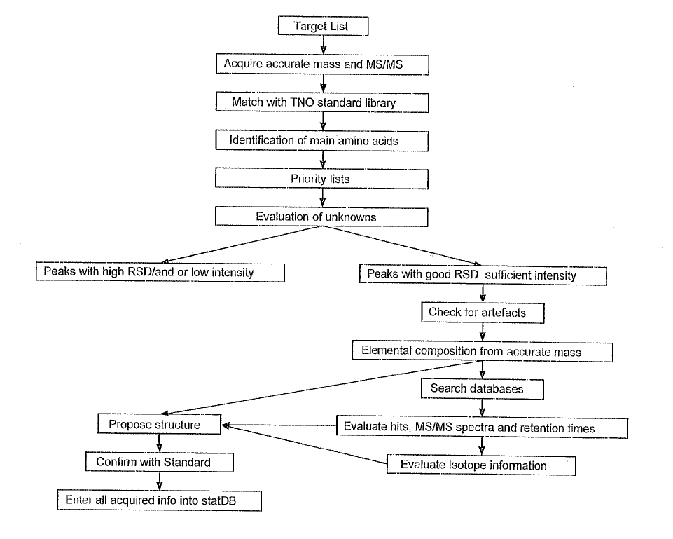

A general overview of all the steps taken to identify analyte peaks that were statistically significant is given in FIG. 2. Accurate mass measurements using Fourier transform mass spectrometry (FTMS) and MS/MS spectra were acquired for all ion peaks of statistical significance if they were of sufficient intensity.

The first identification step was matching of the target compound list with the reference standard database containing a number of amino acids and related compounds. Retention times, accurate masses and, as necessary, MS/MS spectra were compared between the study-specific compounds and those in the reference database.

The remaining target analyte peaks were identified only after they were listed on the priority lists derived from the statistical analysis of these data. The prioritized unknowns were evaluated by checking the raw data and the quality control results. In this identification approach, retention time, accurate mass and MS/MS data were used. Accurate mass experiments were performed on the LTQ-FTMS instrument. The detection of the ions was performed in the FTMS. Resolution at m/z 400 is 100,000. Based on accurate mass and the knowledge about the derivatization used, possible elemental compositions were searched for in the KEGG, Merck and ChemFinder databases. The possible matches were evaluated and for this purpose, the retention times and the MS/MS spectra were used. In some cases, individual compound standards were purchased to confirm identification.

### Statistical Analysis:

For detecting differences in the levels of analytes amongst the groups of rats, a linear model (One-Way ANOVA) was fitted. This model can be parameterized as:

y=β₀+β₁·I_{Treatment=Fenofibrate)}+β₂·I_{(Treatment=MPC-1)}+ε,

where y denotes the analyte to be tested, I is an indicator variable and ε is the error. Under this parameterization, the tests for markers of treatment were as follows:
H₀⁽¹⁾:β₁=0 , testing for the difference between the mean of the fenofibrate group and the mean of the vehicle group; and,
H₀⁽²⁾:β₂=0, testing for the difference between the mean of the MPC-1 group and the mean of the vehicle group.

Because in each analysis tens or hundreds of individual tests were performed simultaneously, some tests were likely to have significant results by chance alone, resulting in false discoveries. In order to control the false discovery rate (FDR) among all significant findings, the method described by Storey (J. R. Statist. Soc. B. 64:479-98 (2002)) was applied.

In each analysis an FDR of 0.15 was allowed; namely in each analysis, approximately 15% of the analytes reported as significant are estimated to be falsely reported. In addition, each test was required to have an unadjusted p-value of at most 0.05 in order to be reported as significant. These parameters are referred to as the default significance parameters.

### Results:

A systems pharmacology approach was employed (van der Greef & McBurney, Nature Rev. Drug. Disc. 4:961-967 (2005)) with plasma samples in a 28-day dosing study of the action of two PPARα agonists, fenofibrate and a development-stage compound MPC-1 (Mitsubishi Pharma Corporation), in rats. The datasets were inspected and the results were ranked on the basis of median fold change compared to vehicle-treated rats using two bioanalytical platforms, Polar LC/MS and GC/MS (van der Greef, et al., J. Proteome Res. 4:1540-59 (2007)). Sarcosine was identified as the analyte with the largest median fold change caused by either drug and measured on both bioanalytical platforms. At the end of the 28-day treatment period, the median fold change in plasma sarcosine abundance resulting from daily treatment with 300 mg/kg fenofibrate relative to treatment each day with the vehicle was 4.25 as measured by the Polar LC/MS platform and 3.74 as measured by the GC/MS platform. At the end of the 28-day treatment period, the median fold change in plasma sarcosine abundance resulting from daily treatment with 15 mg/kg MPC-1 relative to treatment each day with the vehicle was 6.51 as measured by the Polar LC/MS platform and 5.83 as measured by the GC/MS platform. The good agreement of the results across both platforms and both compounds is consistent with the elevation of plasma sarcosine being a general result of treatment of a mammal with a PPARα agonist.

Treatment of rats for 28-days with fenofibrate at 100 mg/kg/day further demonstrated that the PPARα agonist effect on plasma sarcosine levels was a dose-dependent phenomenon.

The observed increases in plasma sarcosine levels resulting from administration of PPARα agonists are consistent with the levels of sarcosine required for substantial inhibition of the GLYT 1 glycine transporter and effective treatment of schizophrenia.

As the concentration of sarcosine in blood serum of normal human subjects is reported to be 1.59±1.08 μM, such plasma median fold elevations of sarcosine concentration caused by the PPAR-alpha agonists would be consistent with a substantial inhibition of the GLYT1 glycine transporter. Such an inhibition of GLYT1 would be expected to elevate glycine concentrations in the region of NMDA receptors and increase NMDA receptor activation by the neurotransmitter glutamate. In schizophrenic patients, and patients suffering from other psychiatric or neurological diseases or disorders associated with NMDA receptor hypofunction, the increased NMDA receptor activation would be expected to improve symptoms and prognosis.

Having described certain embodiments of the invention, it will be apparent to those of ordinary skill in the art that other embodiments incorporating the concepts disclosed herein may be used without departing from the spirit and scope of the invention. The described embodiments are to be considered in all respects as only illustrative and not restrictive.

What is claimed is:

1. A method of increasing sarcosine levels in a patient in need thereof, the method comprising activating peroxisome proliferator activated receptor type a (PPARα) in the patient.

2. A method of promoting N-methyl-D-aspartate (NMDA) receptor activation in a patient in need thereof, the method comprising increasing sarcosine levels in the patient according to the method of claim 1.

3. A method of treating schizophrenia in a patient in need thereof, the method comprising promoting NMDA receptor activation in the patient according to the method of claim 2.

4. The method of claim 3, further comprising antagonizing dopamine D2 receptors in the patient.

5. The method of claim 1, comprising administering a compound selected from the group consisting of clofibrate, gemfibrozil, ciprofibrate, bezafibrate, fenofibrate, simfibrate, and clofibride, and pharmaceutically acceptable salts thereof.

6. The method of claim 2, comprising administering a compound selected from the group consisting of clofibrate, gemfibrozil, ciprofibrate, bezafibrate, fenofibrate, simfibrate, and clofibride, and pharmaceutically acceptable salts thereof.

7. The method of claim 3, comprising administering a compound selected from the group consisting of clofibrate, gemfibrozil, ciprofibrate, bezafibrate, fenofibrate, simfibrate, and clofibride, and pharmaceutically acceptable salts thereof.

8. The method of claim 4, comprising administering a compound selected from the group consisting of clofibrate, gemfibrozil, ciprofibrate, bezafibrate, fenofibrate, simfibrate, and clofibride, and pharmaceutically acceptable salts thereof.
